(19) United States
(12) Patent Application Publication
(10) Pub. No.: US 2025/0370124 A1
(43) Pub. Date: Dec. 4, 2025

(54) SYSTEM AND METHOD FOR DETECTING PRESENCE OF BODIES IN VEHICLES

(71) Applicant: VAYYAR IMAGING LTD., Yehud (IL)

(72) Inventors: Michael ORLOVSKY, Hod Ha-Sharon (IL); Shahar KATZ, Tel Aviv (IL); Shachar RESISI, Shoresh (IL)

(21) Appl. No.: 18/707,910

(22) PCT Filed: Nov. 10, 2022
(86) PCT No.: PCT/IB2022/060820

§ 371 Date: May 7, 2024

Related U.S. Application Data
(60) Provisional application No. 63/277,673, filed on Nov. 10, 2021.

(51) Int. Cl. G01S 13/89; G06T 7/246; G08B 21/18

(52) U.S. Cl. CPC G01S 13/89; G06T 7/246; G06T 2200/04; G06T 2207/20072; G06T 2207/20224; G06T 2207/30196; G06T 2207/30268; G08B 21/18

(57) ABSTRACT

Systems and methods for detecting living bodies in vehicles and generating alerts only if a child is detected. A radar detection system uses vehicle vibration, temporal behavior analysis and spatial characteristics modules to detect false positives by analyzing image data over time and space to distinguish between real children and other similar voxel clusters within the radar images.

### CROSS-REFERENCE TO RELATED APPLICATIONS

This application claims the benefit of priority from 63/277,673 filed on Nov. 10, 2021 the contents of which is incorporated by reference in its entirety.

### FIELD OF THE DISCLOSURE

The disclosure herein relates to systems and methods detecting presence of living bodies in vehicles. In particular the invention relates to alerting third parties to the presence of an infant or other dependent accidently forgotten in the cabin.

### BACKGROUND

Child Present Detection is an assessment protocol directed towards reducing risks to victims in situations where an infant or child is trapped in a vehicle. Such a protocol may also provide alarms in the cases of pets or other dependents which may be left in closed cabins.

Whilst the need for providing alerts and alarms is necessary in every case that a life is at risk from abandonment, the accuracy of such alarms is essential in order to prevent excessive false alarms. Excessive false alarm rates present a genuine life risk because they lead to alert fatigue. Those subject to excessive alerts are likely to ignore or even deactivate future alerts.

Furthermore, where there is high confidence in the accuracy of a detection, active interventions may be taken to mitigate risk. For example, vehicle windows may be opened, an air conditioner activated or the like.

The need remains, therefore, for a child presence detection system with high true positive detection rate and low false positive detection rate. The invention described herein addresses the above-described needs.

### SUMMARY OF THE EMBODIMENTS

According to one aspect of the current invention, a system is introduced for detecting the presence of bodies in a vehicle cabin. The system includes a radar unit comprising at least one transmitter antenna connected to an oscillator and configured to transmit electromagnetic waves into the vehicle cabin, and at least one receiver antenna configured to receive electromagnetic waves reflected by objects within the vehicle cabin and operable to generate raw data.

The system further includes a processor unit configured to receive data from the radar unit and operable to generate alert instructions based upon the data; and an alert generator configured to generate child present detection (CPD) alerts.

The processor typically comprises modules such as a vehicle vibration detection module operable to detect vibrations of the vehicle cabin; a temporal behavior analysis module operable to analyze temporal characteristics of movements and to identify oscillations characteristic of breathing; and a spatial characteristic analysis module operable to analyze spatial features of clusters of voxels detected within the vehicle cabin.

The system may further include a communication module configured and operable to communicate child present detection alerts to third parties possibly in communication with a computer network.

Where appropriate, the system may further include a preprocessor configured to receive raw data from the radar unit and to operable to produce a filtered point cloud for model optimization. Additionally or alternatively, the system includes a frame buffer memory unit configured and operable to store frame data.

Another aspect of the current invention is to teach a method for detecting the presence of bodies in a vehicle cabin. The method may include steps of: providing a radar module; providing a vehicle vibration detection module; providing a temporal behavior analysis module; providing a spatial characteristic analysis module; and providing presence detection unit. Accordingly, the method may further include at least one transmitter antenna transmitting electromagnetic waves into the vehicle cabin; at least one receiver antenna receiving electromagnetic waves reflected by objects within the vehicle cabin; transferring data from radar to processor; the vehicle vibration detection module generating a vehicle vibration index; the temporal behavior analysis module generating temporal movement indices; the spatial characteristic analysis module generating spatial feature indices; transferring a feature vector to the presence detection unit; the presence detection unit processing the feature vector; and if a body is detected then providing an alert.

Where appropriate, the step of the vehicle vibration detection module generating a vehicle vibration index may comprise: obtaining a series of three dimensional frames of image data; removing static objects from the image data; generating a two dimensional moving target indication matrix; and summing the lowest intensity values of pixels in the two dimensional moving target indication matrix.

Optionally, the step of removing static objects from the image data may include: selecting a frame capture rate; collecting raw data from a first frame; waiting for a time delay; collecting raw data from a second frame; and subtracting the first frame data from the second frame data.

Optionally again, the step of generating a two dimensional moving target indication matrix may include: identifying a maximum intensity voxel (rₘₐₓ, θ, φ) for each pair of angular coordinates (θ, φ); and constructing a two dimensional matrix with each pixel (θ, φ) assigned a value Imax equal to the intensity of the identified maximum voxel.

In various embodiments, the step of the temporal behavior analysis module generating temporal movement indices compises: identifying clusters of high intensity voxels within three dimensional image data; for each cluster, the processor unit collating a series of complex values for each voxel; for each voxel determining a center point in the complex plane; determining a phase value for each voxel in each frame; generating a smooth waveform representing phase changes over time for each voxel in each frame; selecting a subset of voxels indicative of a breathing pattern; and calculating temporal movement indices.

Optionally, the step of calculating temporal movement indices comprises calculating a spectral peak index, a respiration per minute (RPM) index, or a circle fit index.

Additionally or alternatively, the step of the spatial characteristic analysis module generating spatial feature indices comprises: obtaining a series of three dimensional frames of image data of an arena including the vehicle cabin and the surroundings; identifying voxel clusters within the arena; counting the clusters within the target region thereby obtaining a cluster number index; counting the number of voxels in each cluster; selecting the largest number of voxels thereby obtaining a max-cluster size index; obtaining a cluster depth index; obtaining a target-max voxel index; obtaining an arena-max voxel index; and obtaining a max-voxel range index.

Variously, the step of obtaining a cluster depth index may include calculating the difference between the maximum range and the minimum range of voxels within each cluster; and selecting the value closest to an infant reference value. The step of obtaining a target-max voxel index may comprise selecting the highest moving target indication value within the arena. The step of obtaining an arena-max voxel index may comprise selecting the highest MTI value within the arena. Further the step of obtaining a max-voxel range index may comprise selecting the range of the arena-max voxel.

In other embodiments of the invention, the method may further comprise distinguishing between a child and a pet. For example the method may include: identifying a child sized target; transmitting a pet stimulation signal insignificant to humans; if increased activity is detected in the child sized target then associating the child sized target with a pet. Accordingly, where appropriate, the step of transmitting a pet stimulation signal insignificant to humans comprises transmitting a pet stimulation signal at a frequency inaudible to human ears.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1A is a block diagram schematically representing selected components of a system for detecting the presence of bodies in vehicles;

FIG. 1B is a flowchart schematically representing data flow between components of the system for detecting the presence and alerting of bodies in vehicles;

FIG. 1C is a flowchart schematically representing selected actions in a method for detecting the presence of bodies in vehicles;

FIG. 2A is flowchart schematically representing a possible method for generating a vehicle vibration index;

FIG. 2B is flowchart schematically representing possible steps for removing static objects from the image data;

FIG. 2C is a flowchart schematically representing possible steps for generating an MTI matrix;

FIG. 3A illustrates a segment of a three dimensional image illustrating a selected voxel;

FIG. 3B illustrates a set of voxels sharing the same angular coordinates and having different r coordinates;

FIG. 3C an example of a 2D MTI image matrix;

FIG. 3D illustrates an example of an ordered MTI intensity profile showing a typical MTI profile characteristic of a moving object in a stationary environment;

FIG. 3E illustrates an example of an ordered MTI intensity profile showing a typical MTI profile characteristic of a vibrating environment;

FIG. 4 is a flowchart schematically representing a possible method for generating the temporal movement indices;

FIGS. 5A-F indicate examples of plots of series of complex values representing reflected radiation at single voxels within a target region over multiple frames;

FIG. 6 is a flowchart schematically representing a possible method for generating the spatial feature indices;

FIG. 7A is a flowchart schematically representing a possible method for pet mitigation; and

FIG. 7B is a flowchart schematically representing how either Activity Level (AL) or Bounding Volume (BV) covered by a cluster of pixels may be used to indicate the presence of a pet such as a dog.

### DETAILED DESCRIPTION

Aspects of the present disclosure relate to systems and methods for detecting presence of living bodies in vehicles and generating alerts. In particular the invention relates to a child presence detection system with high true positive detection rate and low false positive detection rate.

It has been found that false alarms in detection systems are often caused by objects placed in the passenger cabin or cabin state which meet the trigger conditions of the sensor resulting in an alert without a real infant or child being in the cabin.

A number of false alarm trigger conditions have been identified, for example, a water bottle in a car cabin shaken by wind, by hand or by any other means, may generate an oscillating signal which is superficially similar to a breathing child. Accordingly, a vehicle vibration detection module may apply various methods, as disclosed herein, to allow a shaken vehicle to be detected. In this manner, a shaken vehicle type false alarm trigger may be averted.

Another false alarm trigger may be an oscillating object such as a pendulum, a spring, a clock or the like which are typically characterized by a very periodic movement. Accordingly, a temporal behavior analysis module may be provided to analyze the temporal characteristics of the movements to identify those oscillations which are characteristic of real breathing.

Still other characteristics may be used to distinguish between true and false alarms for example spatial features relating to the size and shape of the suspected child. Accordingly, a spatial characteristic analysis module may be provided to analyze features of clusters of voxels detected within the vehicle cabin in order to identify clusters indicative of real children or the like.

It is further noted that even when a real living body is detected within the vehicle, this living body may be a pet such as a dog a cat or the like. Accordingly, a Pet Mitigation Module may be provided to distinguish pets from humans when required.

As required, detailed embodiments of the present invention are disclosed herein; however, it is to be understood that the disclosed embodiments are merely examples of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

As appropriate, in various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally, or alternatively, the data processor may access a non-volatile storage, for example, a magnetic hard-disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting. Accordingly, various embodiments may omit, substitute, or add various procedures or components as appropriate. For instance, the methods may be performed in an order different from described, and that various steps may be added, omitted or combined. In addition, aspects and components described with respect to certain embodiments may be combined in various other embodiments.

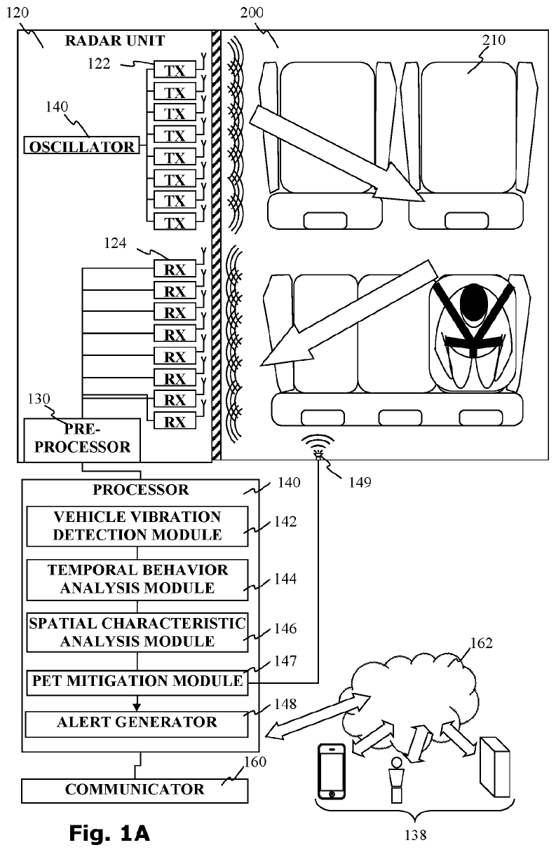

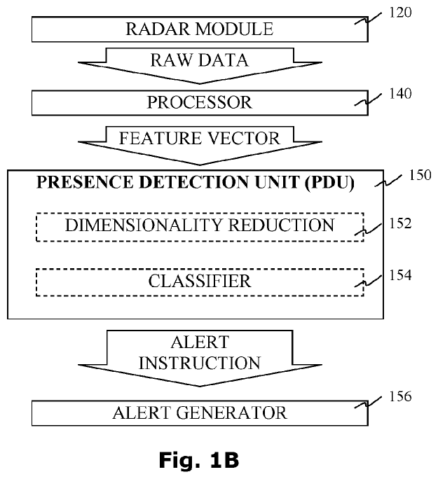

Reference is now made to the block diagram of FIG. 1A which schematically representing selected components of a system 100 for detecting the presence of bodies in vehicles. The system 100 includes a radar unit 120 and a processor 140.

The radar 120 typically includes at least one array of radio frequency transmitter antennas 122 and at least one array of radio frequency receiver antennas 124. The radio frequency transmitter antennas are connected to an oscillator 126 (radio frequency signal source) and are configured and operable to transmit electromagnetic waves towards the target region 200. The radio frequency receiver antennas 124 are configured to receive electromagnetic waves reflected back from objects 210 within the target region 200.

Accordingly the transmitter may be configured to produce a beam of electromagnetic radiation, such as microwave radiation or the like, directed towards a monitored region 200 such as vehicle cabin or the like. The receiver may include at least one receiving antenna or array of receiver antennas configured and operable to receive electromagnetic waves reflected by objects within the monitored region.

The raw data generated by the receivers is typically a set of complex values indicative of magnitude and phase measurements corresponding to the waves scattered back from the objects in front of the array. Spatial reconstruction processing is applied to the measurements to reconstruct the amplitude (scattering strength) at the three dimensional coordinates of interest within the target region. Thus each three dimensional section of the volume within the target region may represented by a voxel defined by four values corresponding to an x-coordinate, a y-coordinate, a z-coordinate, and an amplitude value.

Typically the receivers are connected to a pre-processing unit 130 configured and operable to process the amplitude matrix of raw data generated by the receivers and which may produce a filtered point cloud suitable for model optimization.

Accordingly, where appropriate, a preprocessing unit may include an amplitude filter operable to select voxels having amplitude above a required threshold and a voxel selector operable to reduce the number of voxels in the filtered data, for example by sampling the data or clustering neighboring voxels. In this manner the filtered point cloud may be output to a processor. It is further note that the filtered point cloud may further be simplified by setting the amplitude value of each voxel to ONE when the amplitude is above the threshold and to ZERO when the amplitude is below the threshold.

The processor 140 which is in communication with the preprocessor unit may include modules such as a vehicle vibration detection module 142, a temporal behavior analysis module 144, a spatial characteristic analysis module 146, optionally a pet mitigation module 147 and an alert generator 148 which may be configured to receive a feature vector including a combination of feature indices generated by the analysis modules and operable to generate child present detection (CPD) alerts based upon the received data.

A communication module 160 is configured and operable to communicate child present detection alerts to third parties. Optionally the communication module 160 may be in communication with a computer network 162 such as the internet via which it may communicate alerts to third parties for example via telephones, computers, wearable devices or the like.

In still other embodiments, the CPD alert may initiate active interventions may be taken to mitigate risk, for example, vehicle windows may be opened, an air conditioner activated or the like.

With reference now to the flowchart of FIG. 1B which indicates how data may flow between components of the systems in order to generate CPD alerts. The radar module 120 may produce raw data which is passed to the processor 140 which generates a feature vector. The feature vector is used by a presence detection unit 150. The presence detection unit 150 is operable to decide whether a child is really present and to communicate an alert instruction to the alert generator 156 where appropriate. The presence detection unit 150 may include a dimensionality reduction unit 152, operable to convert the multidimensional feature vector for principle component analysis, and a classifier 154 such as a support vector machine operable to classify the feature vector into either presence-detected or NOT-presence-detected.

Reference is now made to the flowchart of FIG. 1C which schematically represents selected actions in a method for detecting the presence of bodies in vehicles. The method includes providing a radar module 1001, providing a vehicle vibration detection module 1002, providing a temporal behavior analysis module 1003, proving a spatial characteristic analysis module 1004 and providing a presence detection unit (PDU) 1005.

Accordingly, alerts may be generated by the radar scanning the target region 1006 and transferring raw data to the processor 1007, the vehicle vibration detection module generating a vibration index 1008, the temporal behavior analysis module generating temporal movement indices 1009, such as a spectral peak index, an RPM index and a circle fit index or the like, and the spatial characteristic analysis module generating spatial feature indices 1010.

The method may continue with the processor transferring a feature vector including these indices to the presence detection unit 1011, the presence detection unit processes the feature vector 1012 and deciding if a living body is detected 1013.

If no body is detected 1014 the radar continues to scan the target region 1006. If a body is detected then an alert is generated 1015 and the radar also continues to scan the target region 1006 as before.

Where required, an additional step of applying pet mitigation 1016 may be included so as to distinguish between human and animal bodies.

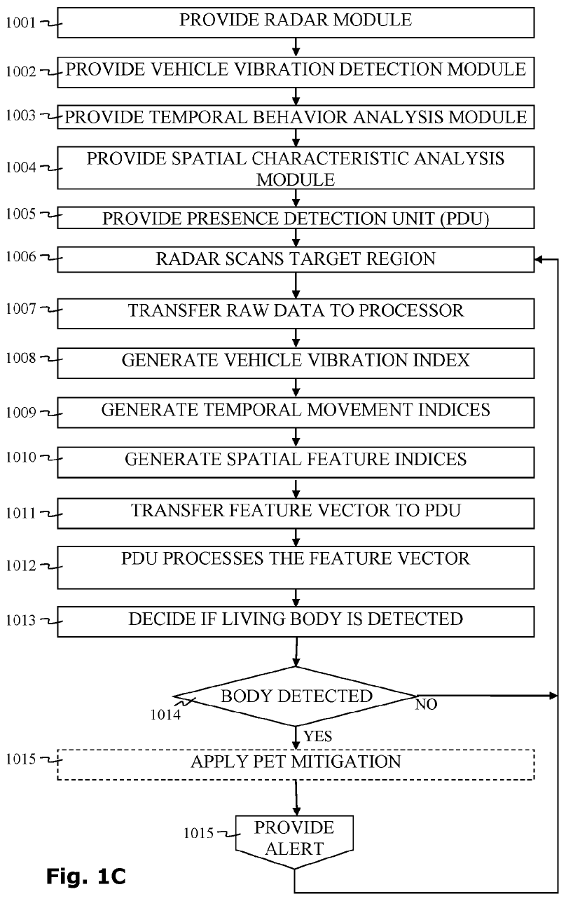

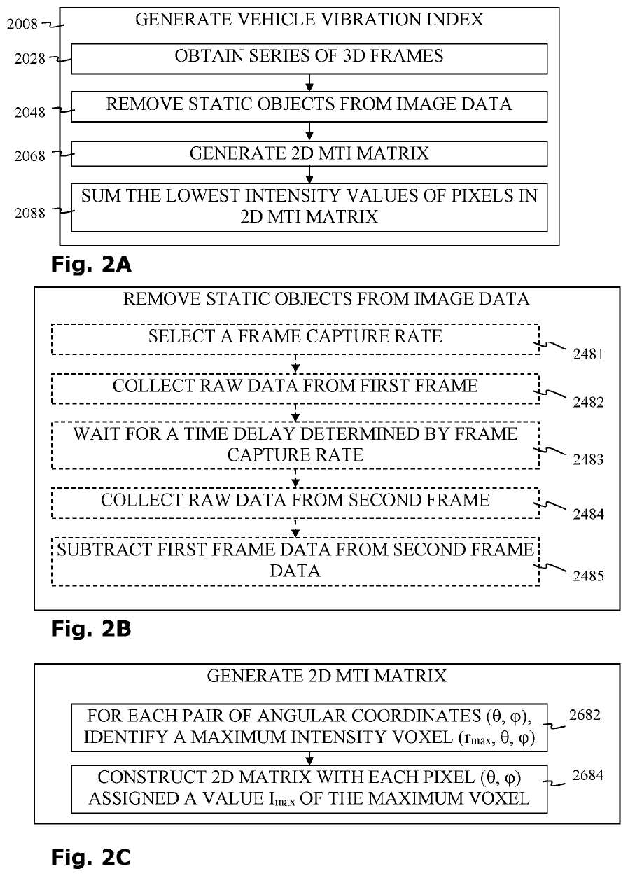

Referring now to FIG. 2A a flowchart is presented which schematically represents a possible method for generating a vehicle vibration index 2008 as described above. Optionally, the vehicle vibration detection module obtains a series of three dimensional frames representing radar images captured of the target region 2028, removes static objections from the image data 2048 thereby generating a two dimensional Moving Target Indication (MTI) matrix 2068, accordingly, the lowest intensity values of pixels, say the lowest five percent values, in the MTI matrix may be summed 2088 thereby providing an indication of the background movement of the target region.

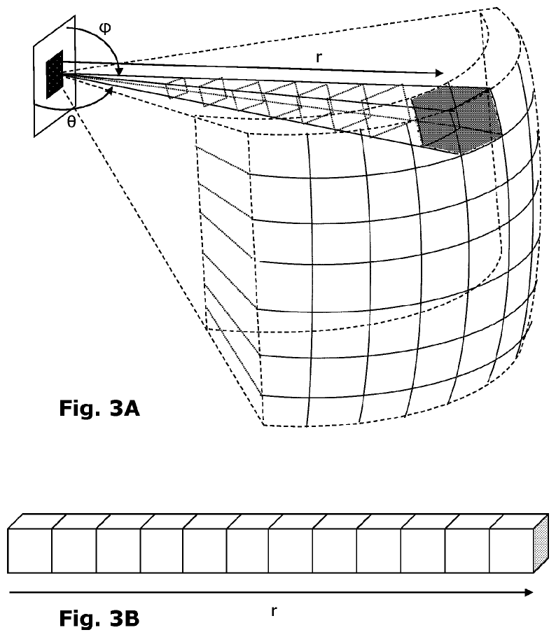

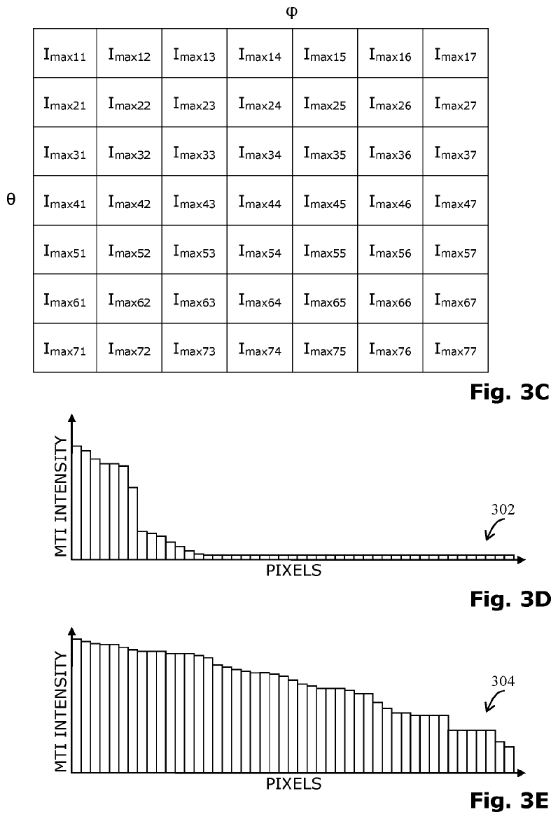

As indicated in the MTI intensity profile of FIG. 3D, it is expected that the lowest intensity pixels 302 in a stationary vehicle should be very low. However, as indicated in the MTI intensity profile of FIG. 3E, it is expected that the lowest intensity pixels in a shaking vehicle may be much higher 304.

The sum of the lowest intensity pixels of the MTI matrix may serve as an effective vehicle vibration index.

A possible way for removing static objects from the image data 2048 is represented in the flowchart of FIG. 2B. A temporal filter may be applied to select a frame capture rate 2481, to collect raw data from a first frame 2482; to wait for a time delay 2483, perhaps determined by frame capture rate; to collect raw data from a second frame 2484; and to subtract first frame data from the second frame data 2485. In this way a filtered image may be produced from which static background is removed and the only moving target data remain.

By storing multiple frames within a frame buffer memory unit, the temporal filter may be further improved by applying a Moving Target Indication (MTI) filter such as described in the applicant's copending International Patent Application No. PCT/IB2022/055109 which is incorporated herein in its entirety.

An MTI may be applied to data signals before they are transferred to the image reconstruction block or directly to the image data. MTI may estimate background data for example using an infinite impulse response (IIR) low-pass filter (LPF). This background data is subtracted from the image data to isolate reflections from moving objects. It is noted that such a process may be achieved by subtracting the mean value of several previous frames from the current frame. Optionally, the mean may be calculated by an IIR or an FIR low-pass filter such as the above described LPF implementation.

The MTI IIR filter time constant, or the duration over which the average is taken by the IIR response is generally fixed to best suit requirements, either short to better fit dynamic targets or long to fit still or slow targets.

Accordingly, the MTI method may include steps such as selecting a filter time constant, applying an IIR filter over the duration of the selected time constant, applying a low pass filter, and removing the background from the raw data.

It has been found that MTI may generate artifacts such as phantoms when objects are suddenly removed from the background. For example, when a chair is moved, a person moves in their sleep, a wall is briefly occluded, of the like, subsequent background subtraction may cause such events to leave shadows in the image at the previously occupied location. Since signals are complex, it is not possible to distinguish between a real object and its negative shadow.

Similarly, obscured stationary objects in the background may appear to be dynamic when they suddenly appear when uncovered by a moving object in the foreground.

Furthermore, slow changes of interest may be repressed, for example the reflections from people sitting or lying still may change little over time and thus their effects may be attenuated by background subtraction.

Accordingly, a three dimensional MTI array may be generated from which a two dimensional MTI matrix may be generated for example as described in FIG. 2C. A two dimensional matrix may be generated, for example, by identifying a maximum intensity voxel (rₘₐₓ, θ, φ) 2682, for each pair of angular coordinates (θ, φ), and constructing a two dimensional matrix with each pixel (θ, φ) 2684 assigned a value Imax of the identified maximum voxel.

As illustrated in FIG. 3A, the three dimensional MTI array may comprise a three dimensional array of voxels, each voxel being characterized by a set of spherical coordinates (r, θ, φ) and an associated value of amplitude of energy reflected from those polar coordinates. For the purposes of illustration let the MTI intensity value of each voxel be given by the function I(r, θ, φ) where r is the radial distance r to the voxel from the radar, θ is the polar angle towards the voxel, and φ is the azimuthal angle towards the voxel.

The three dimensional MTI array of the target region is reduced to a two dimensional MTI matrix by constructing a matrix comprising a two dimensional array of pixels. Accordingly, a unique MTI value I(θ, φ) is selected for each pixel characterized by a pair of angular coordinates (θ, φ).

It is noted that although a spherical coordinate system is described herein, equivalent methods may use other three dimensional coordinate systems, such as cylindrical coordinates, cartesian coordinates or the like.

FIG. 3B illustrates a set of voxels sharing the same angular coordinates and having different r coordinates. The MTI value may be selected of the voxel with highest MTI value for the associated pair of angular coordinates (θ, φ) regardless of the value of r.

An example of such a two-dimensional MTI matrix is provided in FIG. 3C. Having constructed the two dimensional MTI matrix an MTI intensity distribution profile may be generated by ordering the pixels starting with the pixel having the highest MTI intensity and proceeding to pixels with lower and lower MTI intensity.

FIG. 3D illustrates an example of an ordered MTI intensity profile showing a typical MTI profile characteristic of a moving object in a stationary environment. It is noted that most of the pixels show no movement at all with only the pixels indicating the moving object having high MTI value.

By contrast, FIG. 3E illustrates an example of an ordered MTI intensity profile showing a typical MTI profile characteristic of a vibrating environment, in which vibrations are detected uniformly in all direction. It is noted that all pixels now indicate movement as indicated by the relative uniformity of the MTI profile. This profile would be expected in a shaking vehicle.

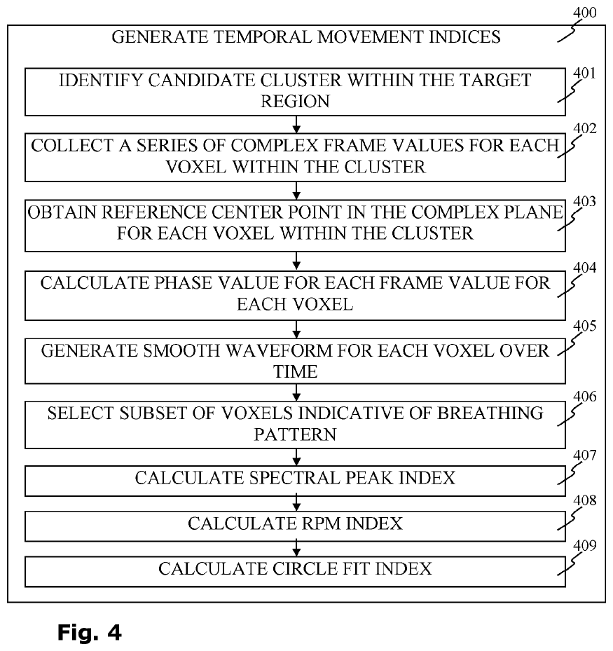

Reference is now made to the flowchart of FIG. 4 which indicates a possible method 400 for the temporal behavior analysis module to generate the temporal movement indices used by the presence detection unit.

The method includes identifying clusters of high intensity voxels within the three dimensional image data of the target region 401, for each cluster, the processor unit collating a series of complex values 402 for each voxel representing reflected radiation for the associated voxel in multiple frames; for each voxel determining a center point in the complex plane 403; determining a phase value for each voxel in each frame 404; generating a smooth waveform representing phase changes over time for each voxel 405; selecting a subset of voxels indicative of a breathing pattern 406; and calculating temporal movement indices 407, such as a spectral peak index, a respiration per minute (RPM) index 408, and a circle fit index 409.

Various values may be selected for the temporal movement indices. By way of example, a spectral peak index may be calculated by taking the ratio between the maximum and mean fast Fourier transforms of the unwrapped phase

$$\frac{max ( F F T ( S ) )}{\overline{FFT ( S )}} .$$

An example of a RPM index may be given number by calculating a value for:

$$\frac{60}{{T}_{F F T}} ⋆ ( arg max ( FFT ( s ) ) − \frac{{N}_{F F T} − 1}{2} ) .$$

Again, by way of example, the circle fit index, which may indicated how closely the complex values fit a circle in the complex plane, may be given by the standard deviation of the ratio of the magnitude of the complex vectors to the maximum magnitude

$$S T D ( \frac{\hat{R}}{max ( \hat{R} )} ) .$$

Optionally, the processor may generate a series of frames, where each frame comprises an array of complex values representing radiation reflected from each voxel of the target region during a given time segment.

In one embodiment, the method of the invention monitors over a time period a plurality of voxels in parallel. The signal received by the receiver may be given by:

$${s}_{v} [ n ] = {A}_{v} + {R}_{v} \cdot exp ( j {ϕ}_{v} + j \frac{4 π}{λ} {B}_{v} w [ n ] ) + {v}_{v} [ n ]$$

where v is an index of the voxels, n is a time index, Aᵥ is the DC part of the voxel, due to leakage and static objects. Rᵥ is the amplitude (or radius) of the phase varying part of voxel v, ϕᵥ is a nuisance phase offset of the voxel v, λ is the wavelength, Bᵥ is the effective displacement magnitude of the voxel v, vᵥ[n] is additive noise, and w[n] is the waveform at time n.

For each monitored voxel, the reference center point Âᵥ is calculated. By way of illustration, a center may be determined according to a linear-mean-square-error estimator of circle center. For example, estimation may be based on moments of the real and imaginary parts of the received signal. The moments can be averaged with an infinite impulse response (IIR) filter. The forgetting factor of the IIR filter has an adaptive control that balances between the need to converge quickly to a new value upon a change in the environment (e.g. movement of the subject) and the need to maintain consistency of the estimation.

A phase value for each voxel in each frame may be determined by the processor collating a series of complex values for each voxel representing reflected radiation for the associated voxel in multiple frames; and for each voxel determining a center point in the complex plane; and calculating the arctan of the ratio of the imaginary component and the real component of the difference between the frame value and the center point.

Accordingly, given a reference center point, the phase of a voxel v at a given time instant n may be calculated as:

$${θ}_{v} [ n ] = ∠ ( {s}_{ν} [ n ] - {\hat{A}}_{v} ) = atan 2 ( 𝒥 \{ {s}_{v} [ n ] - {\hat{A}}_{v} \} , ℛ \{ {s}_{ν} [ n ] - {\hat{A}}_{v} \} )$$

In order to create a smooth waveform representing phase changes over time, phase values may be rounded. The phase may be unwrapped to generate a smooth waveform without discontinuities greater than π according to the formula:

$${\tilde{θ}}_{v} [ n ] = {θ}_{v} [ n ] + 2 π \cdot round ( \frac{{\tilde{θ}}_{v} [ n - 1 ] - {θ}_{v} [ n ]}{2 π} )$$

In another embodiment, phase un-wrapping may be based on prediction of the next phase based on a few previous phases. Such a prediction may be used to lower frame rates and/or improve the resilience to noise while avoiding cycle slips. For example, the following predictor tends to account for phase momentum:

$${\tilde{θ}}_{v}^{Opt 2} [ n ] = {θ}_{ν} [ n ] + 2 π \cdot round ( \frac{( 1 + α ) \cdot {\tilde{θ}}_{v}^{Opt 2} [ n - 1 ] - α \cdot {\tilde{θ}}_{v}^{Opt 2} [ n - 2 ] - {θ}_{v} [ n ]}{2 π} )$$

where 0<α≤1 is a parameter that controls the weighting of momentum (linear progress of phase) versus stability (zero order hold).

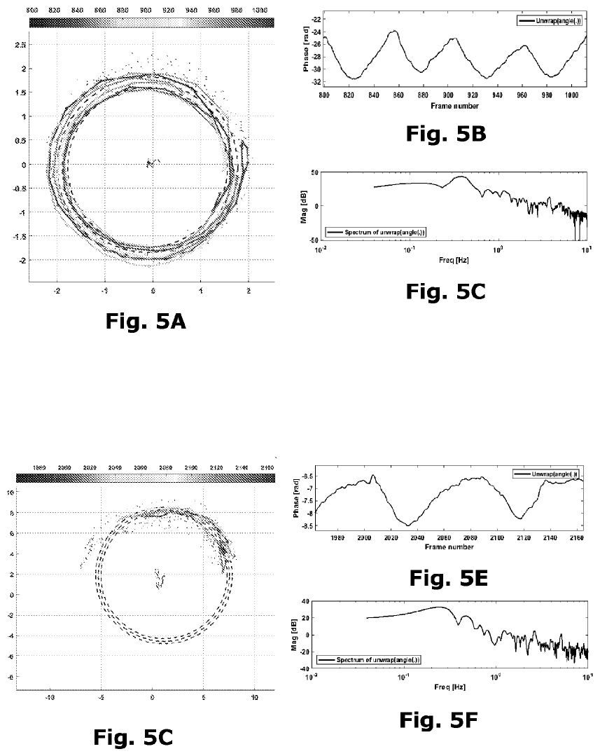

FIGS. 5A and 5D show examples of plots of series of complex values representing reflected radiation at single voxels within a target region over multiple frames. It is noted that the complex values form approximate circles within the complex plane centered at a single point. The periodicity maybe seen over multiple frames giving rise to a characteristic oscillating function such as illustrated in FIGS. 5B and 5E which represent the variation of the phase over time (using frame number as a proxy for time) for the plots of FIGS. 5A and 5B respectively. FIG. 5C and FIG. 5F show corresponding variation in frequency space. Such oscillating functions, may be indicative of breathing or pulse rate for example.

Voxels indicating breathing characteristics and pulse characteristics may be found, for example, by selecting a subset of voxels conforming to selection rules such as using metrics that evaluate the fitness of those voxels. Such metrics may include fitting to the model of arcs of a circle, fitting to predetermined pattern pulse waveform with strong periodicity and the like, and the spatial location of the voxels.

In many cases, the voxels that fit best for breathing tracking are located near the chest and stomach of the breathing person. In other cases, the most adequate selection is other voxels, such as of reflection from walls or ceiling, or movement of other objects due to the breathing.

An arc-fitting metric maybe calculated for the phase values associated with each voxel; and the selected voxels would be those having an arc-fitting metric above a predetermined threshold. For example a metric may evaluate the accuracy of fitting the data to the model described herein. Relative stability may be measured from the distance between the received signal and the estimated reference center point

$$r [ n ] = {s}_{v} [ n ] - {\hat{A}}_{v}$$

The metric may be calculated, for example, as:

$$var ( {\tilde{θ}}_{v} [ n ] ) \cdot \frac{{\overline{r}}^{2}}{var ( r )}$$

Additionally or alternatively, a time dependency function may be calculated for the phase values associated with each voxel; and voxels may be selected which have periodic characteristics indicative the pulse, such as the duration systole, the duration of diastole, pulse rate and the like as well as combinations thereof.

Such a metric may evaluate the fitness of the un-wrapped phase \tilde{θ}ᵥ[n] as a clean pulse waveform. A Fourier transform of this signal may be calculated, and it may be checked that the peak value is achieved at a frequency within the range of reasonable periods expected for breathing or of a normal pulse, and that the energy of this peak divided by average energy in other frequencies.

By way of examples periodic characteristics indicative of breathing may include an inhalation-to-exhalation ratio between say 1:1 and 1:6, a breath rate between say 1 and 10 seconds. Also by way of example, the periodic characteristics indicative of pulse of a subject at rest may include a pulse or heart rate between, say, 45 and 150 beats per minute and a ratio of diastole to systole of about 2:1.

The two metrics above may be smoothed, and then combined into a single metric that represents the fitness of each voxel for extraction of pulse.

In one possible embodiment of this invention, a single voxel is selected for pulse determination, based on the above metrics, with hysteresis to avoid frequent jumping among voxels.

In another embodiment, multiple voxels with high metric values are chosen, and their waveforms are averaged by using SVD (PCA) after weighting by the fitness metric.

Lower frequency oscillations of the phase signal indicative of breathing may be filtered out of the phase profile signal to leave the high phase oscillations indicative of the heart rate.

In still other embodiments, Voxel selection may use further metrics such as signal quality (SNR) to validate that the signal extracted from this voxel would have good enough signal to be useful and Breathing detection to validate that the signal observed is consistent with a real breathing signal. These two metrics may be combined to determine that the voxel is suitable for selection.

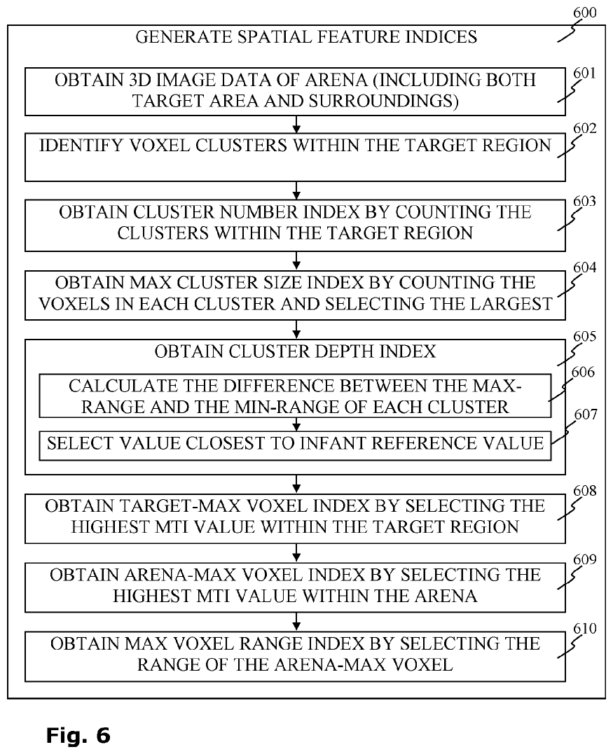

Referring now to the flowchart of FIG. 6, a possible method 600 is presented for generating spatial feature indices. The method includes obtaining three dimensional image data of an arena including the target region as well as its surroundings 601, identifying voxel clusters within the target region 602, counting the clusters within the target region thereby obtaining a cluster number index 603, counting the number of voxels in each cluster and selecting the largest number thereby obtaining a max-cluster size index 604, calculating the difference between the maximum range and the minimum range of voxels within each cluster 606 and selecting the value closest to an infant reference value 607 thereby obtaining a cluster depth index 605, selecting the highest moving target indication (MTI) value within the target region thereby obtaining a target-max voxel index 608, selecting the highest MTI value within the arena thereby obtaining an arena-max voxel index 609, selecting the range of the arena-max voxel thereby obtaining a max-voxel range index 610.

As noted above, where required, a pet mitigation module may be provided to distinguish a pet, such as a dog from a child. Accordingly when a body is detected within the vehicle, pet mitigation may be applied the results of which may determine whether an alert is generated or the nature of such an alert.

Because radar images of some pets may be of similar size and shape to children, pet mitigation may require high resolution imaging to distinguish between them. Moreover, even if using high resolution imaging device, such as cameras, processing time may be too long and automated recognition may be further complicated due to the large variety of different pets and their behavior.

Referring back to FIG. 1A, according to one system the pet mitigation module 147 may be in communication with a pet stimulation signal generator 149 configured to generate a signal to which a pet will typically respond differently to a human. For example, an ultrasonic signal audible to the canine ear and not to the human ear may induce a physical response from a dog which can be detected by the radar 120 while a human body would remain unaffected. Alternative solutions may include tunes or tones to which pets may respond differently to humans in a predictable manner. Such behavioral solutions would be robust and inclusive of many options for pet size, location, posture, and the like.

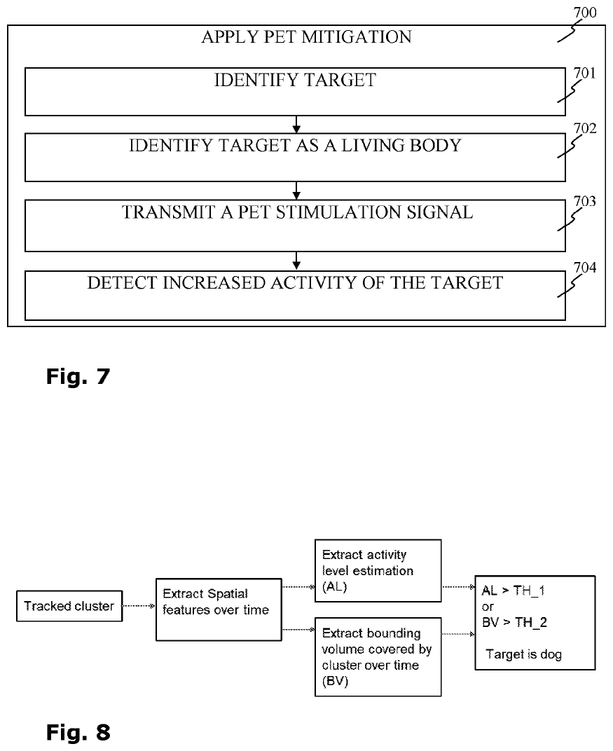

Referring now to FIG. 7 a possible method 700 is presented for applying pet mitigation. A target is identified 701 and identified as a living body 702. A pet stimulation signal may be generated and transmitted into the vehicle cabin 703. The pet stimulation signal is selected such that only pets will react to them. Accordingly, if increased activity of the target is detected 704, such as macromovements between seats and footwells or the like, this activity is indicative of an unrestrained pet within the vehicle cabin rather than a restrained child for example. It will be appreciated that movement of the target may be tracked, for example, by constructing a bounding box around a pixel cluster and tracing the positioning of the center of mass of the bounding box over time. Other possible activity tracking methods may be used as occur to those in the art for example as illustrated in FIG. 8.

### Technical Notes

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

1-5. (canceled)

6. A method for detecting the presence of bodies in a vehicle cabin comprising:
providing a radar module;
providing a vehicle vibration detection module;
providing a temporal behavior analysis module;
providing a spatial characteristic analysis module;
providing presence detection unit;
at least one transmitter antenna transmitting electromagnetic waves into the vehicle cabin;
at least one receiver antenna receiving electromagnetic waves reflected by objects within the vehicle cabin;
transferring data from radar to processor;
the vehicle vibration detection module generating a vehicle vibration index;
the temporal behavior analysis module generating temporal movement indices;
the spatial characteristic analysis module generating spatial feature indices;
transferring a feature vector to the presence detection unit;
the presence detection unit processing the feature vector; and
if a body is detected then providing an alert.

7. The method of claim 6 wherein the step of the vehicle vibration detection module generating a vehicle vibration index comprises:
obtaining a series of three dimensional frames of image data;
removing static objects from the image data;
generating a two dimensional moving target indication matrix; and
summing the lowest intensity values of pixels in the two dimensional moving target indication matrix.

8. The method of claim 7 wherein the step of removing static objects from the image data comprises:
selecting a frame capture rate:
collecting raw data from a first frame;
waiting for a time delay;
collecting raw data from a second frame; and
subtracting the first frame data from the second frame data.

9. The method of claim 7 wherein the step of generating a two dimensional moving target indication matrix comprises:
identifying a maximum intensity voxel (rmax, θ, φ) for each pair of angular coordinates (θ, φ); and
constructing a two dimensional matrix with each pixel (θ, φ) assigned a value Imax equal to the intensity of the identified maximum voxel.

10. The method of claim 6 wherein the step of the temporal behavior analysis module generating temporal movement indices comprises:
identifying clusters of high intensity voxels within three dimensional image data;
for each cluster, the processor unit collating a series of complex values for each voxel;
for each voxel determining a center point in the complex plane;
determining a phase value for each voxel in each frame;
generating a smooth waveform representing phase changes over time for each voxel in each frame; selecting a subset of voxels indicative of a breathing pattern; and
calculating temporal movement indices.

11. The method of claim 10 wherein the step of calculating temporal movement indices comprises calculating a spectral peak index.

12. The method of claim 10 wherein the step of calculating temporal movement indices comprises calculating a respiration per minute (RPM) index.

13. The method of claim 10 wherein the step of calculating temporal movement indices comprises calculating a circle fit index.

14. The method of claim 6 wherein the step of the spatial characteristic analysis module generating spatial feature indices comprises:
obtaining a series of three dimensional frames of image data of an arena including the vehicle cabin and the surroundings;
identifying voxel clusters within the arena;
counting the clusters within the target region thereby obtaining a cluster number index;
counting the number of voxels in each cluster;
selecting the largest number of voxels thereby obtaining a max-cluster size index;
obtaining a cluster depth index;
obtaining a target-max voxel index;
obtaining an arena-max voxel index; and
obtaining a max-voxel range index.

15. The method of claim 14 wherein the step of obtaining a cluster depth index comprises:
calculating the difference between the maximum range and the minimum range of voxels within each cluster; and
selecting the value closest to an infant reference value.

16. The method of claim 14 wherein the step of obtaining a target-max voxel index comprises selecting the highest moving target indication value within the arena

17. The method of claim 14 wherein the step of obtaining an arena-max voxel index comprises selecting the highest MTI value within the arena.

18. The method of claim 14 wherein the step of obtaining a max-voxel range index comprises selecting the range of the arena-max voxel.

19. The method of claim 6 further comprising distinguishing between a child and a pet.

20. The method of claim 6 wherein the step of distinguishing between a child and a pet comprises:
identifying a child sized target;
transmitting a pet stimulation signal insignificant to humans;
if increased activity is detected in the child sized target then associating the child sized target with a pet.

21. The method of claim 6 wherein the step of transmitting a pet stimulation signal insignificant to humans comprises transmitting a pet stimulation signal at a frequency inaudible to human ears.
